(19) United States
(12) Patent Application Publication
(10) Pub. No.: US 2025/0020640 A1
(43) Pub. Date: Jan. 16, 2025

(54) COMPOSITIONS AND METHODS RELATED TO A DUAL-AFFINITY RATIOMETRIC QUENCHING BIOASSAY FOR DETECTING GLYCOPROTEINS

(71) Applicant: North Carolina State University, Raleigh, NC (US)

(72) Inventors: Stefano Menegatti, Raleigh, NC (US); Michael Daniele, Raleigh, NC (US); Brendan Turner, Raleigh, NC (US); Katie Kilgour, Raleigh, NC (US)

(21) Appl. No.: 18/713,047

(22) PCT Filed: Nov. 23, 2022
(86) PCT No.: PCT/US2022/080427

§ 371 Date: May 23, 2024

Related U.S. Application Data
(60) Provisional application No. 63/282,798, filed on Nov. 24, 2021.

(51) Int. Cl. G01N 33/542; G01N 33/569; G01N 33/58

(52) U.S. Cl. CPC G01N 33/542; G01N 33/56983; G01N 33/582; G01N 2333/165; G01N 2469/20

(57) ABSTRACT

The present disclosure provides compositions, methods, and systems related to a dual-affinity ratiometric quenching bioassay. In particular, the present disclosure provides novel compositions and methods that combine selective biorecognition and quenching of fluoescence signals for rapid and sensitive quantification of proteins in complex samples.

### CROSS REFERENCE TO RELATED APPLICATIONS

This application claims priority to and the benefit of U.S. Provisional Patent Application No. 63/282,798 filed Nov. 24, 2021, which is incorporated herein by reference in its entirety for all purposes.

### FIELD

The present disclosure provides compositions, methods, and kits related to a dual-affinity ratiometric quenching bioassay. In particular, the present disclosure provides novel compositions and methods that combine selective biorecognition and quenching of fluorescence signals for rapid and sensitive quantification of proteins in complex samples.

### BACKGROUND

Accurate profiling of post-translational modifications (e.g., glycosylation, phosphorylation, etc.) of target proteins in complex media, such as the serum of patients undergoing immunotherapy, industrial cell culture harvests and streams, and fluids derived from transgenic plants and animals is paramount to ensure the health of patients and product quality in biopharmaceutical industry. To date, a myriad of assays have been developed for antibody detection and measurement, including lateral flow assays, ELISA, and immunoaffinity chromatography. These, however, differ widely in terms of duration (from minutes to hours), performance (sensitivity, limit of detection, and dynamic range), and cost. Likewise, the biorecognition moieties utilized to target the antibody analyte include protein-based affinity tags such as antigens, secondary antibodies and antibody-binding receptors (e.g., Protein A, Protein G, and Fc receptors FcγRs), as well as synthetic affinity tags such as aptamers and peptides. Additionally, detection modalities vary significantly ranging from optical (e.g., UV/vis, fluorescence, and surface plasmon resonance) to electrochemical (e.g., impedance and amperometry) and acoustic (e.g., photoacoustic and quartz crystal microbalance). Fluorescence holds a preeminent place among detection modalities, owing to its high sensitivity, flexibility, and availability of fluorescence spectrophotometers. The generation of a fluorescence signal by the affinity tags can be accomplished either by chemical conjugation (e.g., by labeling them with synthetic fluorophores), or enzymatically by fusing them with enzymes (e.g., horseradish peroxidase or luciferase) that convert substrates into fluorescent products. Of major interest are combinations of fluorophores and labelling strategies that can engage in phenomena such as static or dynamic quenching and energy transfer for the detection of target proteins, including glycoproteins.

### SUMMARY

Embodiments of the present disclosure include a ratiometric bioassay method for detecting a target antibody in a sample. In accordance with these embodiments, the method includes combining an antigen, or fragment thereof, coupled to a first fluorophore and an antibody binding moiety coupled to a second fluorophore with a sample comprising or suspected of comprising a target antibody; exposing the antigen, or fragment thereof, coupled to the first fluorophore, the antibody binding moiety coupled to the second fluorophore, and the sample to light comprising an excitation wavelength of the first and/or the second fluorophore; and detecting emission from the first and/or the second fluorophore. In some embodiments, the emission from the first and the second fluorophore is altered, and wherein the altered emission is proportional to the antibody concentration in the sample.

In some embodiments, the target antibody is capable of binding the antigen.

In some embodiments, the antibody binding moiety is capable of binding a region of the target antibody that does not interfere with the antigen binding. In some embodiments, the antibody binding moiety comprises Protein L, Protein A, Protein G or an antibody-binding fragment thereof.

In some embodiments, the first fluorophore is a high-energy fluorophore and the second fluorophore is a low-energy fluorophore. In some embodiments, the second fluorophore is a high-energy fluorophore and the first fluorophore is a low-energy fluorophore.

In some embodiments, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

In some embodiments, the low-energy fluorophore is selected from the group consisting of Rhodamine, Rhodamine B. Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

In some embodiments, the antibody binding moiety is coupled to Rhodamine.

In some embodiments, the antigen is coupled to fluorescein.

In some embodiments, the method further comprises determining the concentration of the target antibody in the sample based on the altered emission of the first and the second fluorophore. In some embodiments, the altered emission comprises decreased emission, increased emission, and/or a change in emission lifetime.

In some embodiments, the method further comprises incubating the sample comprising or suspected of comprising the target antibody, the antigen coupled to the first fluorophore, and the antibody binding moiety coupled to the second fluorophore for 30 minutes or less prior to measuring the emission of the first and/or the second fluorophore.

In some embodiments, the antigen is coupled to the first fluorophore at an antigen-to-fluorophore ratio of about 1:0.2 to about 1:20. In some embodiments, the antibody binding moiety is coupled to the second fluorophore at a moiety-to-fluorophore ratio of about 1:0.2 to about 1:20. In some embodiments, the first fluorophore and the second fluorophore are present in the composition at a first-to-second fluorophore ratio of about 1:1 to about 1:20.

In some embodiments, the sample is undiluted. In some embodiments, the sample is a whole blood sample, a plasma sample, a serum sample, a urine sample, a saliva sample, or a tissue sample. In some embodiments, sample is a plant sample, a soil sample, a water sample, a cell culture sample, a cell lysate sample, or a cell culture media sample. In some embodiments, the antigen is from a pathogenic organism. In some embodiments, the pathogenic organism is a bacteria, a virus, or a fungus. In some embodiments, the antigen is from SARS-CoV-2.

Embodiments of the present disclosure also include a composition for performing a ratiometric bioassay to detect a target glycoprotein in a sample. In accordance with these embodiments, the composition includes an antigen coupled to a first fluorophore; an antibody binding moiety coupled to a second fluorophore; and a sample comprising or suspected of comprising a target antibody. In some embodiments, emission from the first and the second fluorophore is altered upon exposure to light comprising an excitation wavelength of the first and/or the second fluorophore, and the altered emission is proportional to the target glycoprotein concentration in the sample.

In some embodiments of the method, the target antibody is capable of binding the antigen.

In some embodiments of the method, the antibody binding moiety is capable of binding a region of the target antibody that does not interfere with the antigen binding. In some embodiments of the method, the antibody binding moiety comprises Protein L, Protein A, Protein G or an antibody-binding fragment thereof.

In some embodiments of the method, the first fluorophore is a high-energy fluorophore and the second fluorophore is a low-energy fluorophore.

In some embodiments of the method, the second fluorophore is a high-energy fluorophore and the first fluorophore is a low-energy fluorophore.

In some embodiments of the method, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

In some embodiments of the method, the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

In some embodiments of the method, the altered emission comprises decreased emission, increased emission, and/or a change in emission lifetime.

In some embodiments of the method, the antigen is coupled to the first fluorophore at an antigen-to-fluorophore ratio of about 1:0.5 to about 1:20. In some embodiments of the method, the antibody binding moiety is coupled to the second fluorophore at a moiety-to-fluorophore ratio of about 1:0.2 to about 1:20. In some embodiments of the method, the first fluorophore and the second fluorophore are present in the composition at a first-to-second fluorophore ratio of about 1:1 to about 1:20.

In some embodiments of the method, the sample is undiluted. In some embodiments of the method, the sample is a whole blood sample, a plasma sample, a serum sample, a urine sample, a saliva sample, a tissue sample, a plant sample, a soil sample, a water sample, a cell culture sample, a cell lysate sample, of a cell culture media. In some embodiments, the antigen is from a pathogenic organism. In some embodiments of the method, the antigen is from SARS-CoV-2.

Embodiments of the present disclosure also include a kit for performing a ratiometric bioassay to detect a target glycoprotein in a sample. In accordance with these embodiments, the kit includes an antigen coupled to a first fluorophore; an antibody binding moiety coupled to a second fluorophore; and at least one container.

of the method the kit further comprises a buffer and/or instructions for performing the bioassay. of the method the kit further comprises the target antibody, or a fragment or derivative thereof.

Embodiments of the present disclosure include a ratiometric bioassay method for detecting a target glycoprotein in a sample. In accordance with these embodiments, the method includes combining a lectin or fragment thereof coupled to a first fluorophore, a protein binding moiety coupled to a second fluorophore, and a sample comprising or suspected of comprising a target glycoprotein; exposing the lectin coupled to the first fluorophore, the protein binding moiety coupled to the second fluorophore, and the sample to light comprising an excitation wavelength of the first and/or the second fluorophore; and detecting emission from the first and/or the second fluorophore. In some embodiments, the emission from the first and the second fluorophore is altered, and the altered emission is proportional to the protein concentration in the sample.

In some embodiments, the lectin is capable of binding a glycan epitope on the target glycoprotein in the sample.

In some embodiments, the protein binding moiety is capable of binding a region of the target glycoprotein that does not comprise the glycan epitope.

In some embodiments, the protein binding moiety comprises Protein L, Protein A, Protein G or a protein-binding fragment thereof.

In some embodiments, lectin is selected from the group consisting of LTL (Lotus tetragonolobus), PSA (Pisum sativum), LCA (Lens culinaris), UEA-I (Ulex europaeus), AOL (Aspergillus oryzae), AAL (Aleuria aurantia), MAL_I (Maackia amurensis), SNA (Sambucus nigra), SSA (Sambucus sieboldiana), TJA-I (Trichosanthes japonica), PHAL (Phaseolus vulgaris), ECA (Erythrina cristagalli), RCA120 (Ricinus communis), PHAE (Phaseolus vulgaris), DSA (Datura stramonium), GSL-II (Griffonia simplicifolia), NPA (Narcissus pseudonarcissus), ConA (Canavalia ensiformis), GNA (Galanthus nivalis), HHL (Hippeastrum hybrid), ACG (mushroom, Agrocybe cylindracea), TxLCI (Tulipa gesneriana), BPL (Bauhinia purpurea), TJA-II (Tanthes japonica), EEL (Euonymus europaeus), ABA (fungus, Agaricus bisporus), LEL (tomato, Lycopersicon esculentum), STL (potato, Solanum tuberosum), UDA (Urtica dioica), PWM (pokeweed, Phytolacca Americana), Jacalin (Artocarpus integrifolia), PNA (peanut, Arachis hypogaea), WFA (Wisteria floribunda), ACA (Amaranthus caudatus), MPA (Maclura pomifera), HPA (snail, Helix pomatia), VVA (Vicia villosa), DBA (Dolichos biflorus), SBA (soybean, Dolichos biflorus), Calsepa (Calystegia sepium), PTL-I (Psophocarpus tetragonolobus), MAH (Maackia amurensis), WGA (wheat germ, Triticum aestivum), GSL-I A4 (Griffonia simplicifolia), and GSL-I B4 (Griffonia simplicifolia), or a glycan epitope binding fragment thereof.

In some embodiments, the first fluorophore is a high-energy fluorophore and the second fluorophore is a low-energy fluorophore. In some embodiments, the second fluorophore is a high-energy fluorophore and the first fluorophore is a low-energy fluorophore. In some embodiments, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532. In some embodiments, the low-energy fluorophore is selected from the group consisting of Rhodamine, Rhodamine B. Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

In some embodiments, the lectin is coupled to Rhodamine. In some embodiments, the protein binding moiety is coupled to fluorescein.

In some embodiments, the method further comprises determining the concentration of the target glycoprotein in the sample based on the altered emission of the first and the second fluorophore. In some embodiments, the altered emission comprises decreased emission, increased emission, and/or a change in emission lifetime.

In some embodiments, the method further comprises incubating the sample comprising or suspected of comprising the target glycoprotein, the lectin coupled to the first fluorophore, and the protein binding moiety coupled to the second fluorophore for 30 minutes or less prior to measuring the emission of the first and/or the second fluorophore.

In some embodiments, the lectin is coupled to the first fluorophore at a lectin-to-fluorophore ratio of about 1:0.2 to about 1:20.

In some embodiments, the protein binding moiety is coupled to the second fluorophore at a moiety-to-fluorophore ratio of about 1:0.2 to about 1:20.

In some embodiments, the first fluorophore and the second fluorophore are present in the composition at a first-to-second fluorophore ratio of about 1:1 to about 1:20.

In some embodiments, the sample is undiluted. In some embodiments, the sample is a whole blood sample, a plasma sample, a serum sample, a urine sample, a saliva sample, or a tissue sample. In some embodiments, the sample is a plant sample, a soil sample, a water sample, a cell culture sample, a cell lysate sample, or a cell culture media sample.

In some embodiments, the target glycoprotein is an antibody, a blood protein, a mammalian cell surface protein, or a growth factor. In some embodiments, the target glycoprotein is from a bacteria, virus, or fungus.

Embodiments of the present disclosure also include a composition for performing a ratiometric bioassay to detect a target glycoprotein in a sample. In accordance with these embodiments, the composition includes a lectin coupled to a first fluorophore; a protein binding moiety coupled to a second fluorophore; and a sample comprising or suspected of comprising a target glycoprotein. In some embodiments, emission from the first and the second fluorophore is altered upon exposure to light comprising an excitation wavelength of the first and/or the second fluorophore, and the altered emission is proportional to the target glycoprotein concentration in the sample.

In some embodiments, the composition can be used to determine the concentration of the target glycoprotein in the sample based on the altered emission of the first and the second fluorophore. In some embodiments, the altered emission comprises decreased emission, increased emission, and/or a change in emission lifetime.

In some embodiments, the lectin is capable of binding a glycan epitope on the target glycoprotein in the sample.

In some embodiments, the protein binding moiety is capable of binding a region of the target glycoprotein that does not comprise the glycan epitope.

In some embodiments, the protein binding moiety comprises Protein L, Protein A, Protein G, or a protein-binding fragment thereof.

In some embodiments, the first fluorophore is a high-energy fluorophore and the second fluorophore is a low-energy fluorophore. In some embodiments, the second fluorophore is a high-energy fluorophore and the first fluorophore is a low-energy fluorophore.

In some embodiments, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532. In some embodiments, the low-energy fluorophore is selected from the group consisting of Rhodamine, Rhodamine B, Rhodamine Red-X, Tetramethylrhodamine. Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

In some embodiments, the altered emission comprises decreased emission, increased emission, and/or a change in emission lifetime.

In some embodiments, the lectin is coupled to the first fluorophore at a lectin-to-fluorophore ratio of about 1:0.5 to about 1:20.

In some embodiments, the protein binding moiety is coupled to the second fluorophore at an moiety-to-fluorophore ratio of about 1:0.2 to about 1:20.

In some embodiments, the first fluorophore and the second fluorophore are present in the composition at a first-to-second fluorophore ratio of about 1:1 to about 1:20.

In some embodiments, the sample is undiluted. In some embodiments, the sample is a whole blood sample, a plasma sample, a serum sample, a urine sample, a saliva sample, a tissue sample, a plant sample, a soil sample, a water sample, a cell culture sample, a cell lysate sample, of a cell culture media.

In some embodiments, the target glycoprotein is an antibody, a blood protein, a mammalian cell surface protein, a growth factor, or a protein from a pathogenic organism.

Embodiments of the present disclosure also include a kit for performing a ratiometric bioassay to detect a target glycoprotein in a sample. In accordance with these embodiments, the kit includes a lectin coupled to a first fluorophore; a protein binding moiety coupled to a second fluorophore; and at least one container.

In some embodiments, the kit further comprises a buffer and/or instructions for performing the bioassay.

In some embodiments, the kit further comprises the target glycoprotein or a fragment or derivative thereof.

### BRIEF DESCRIPTION OF THE DRAWINGS

FIGS. 1A-1D: Representative results demonstrating SARS-CoV-2 nucleocapsid antibody detection in PBS across the timepoints indicated using the DARQ assays of the present disclosure.

FIGS. 2A-2D: Representative results demonstrating SARS-CoV-2 nucleocapsid antibody detection in saliva samples across the timepoints indicated using the DARQ assays of the present disclosure.

FIGS. 3A-3D: Representative results demonstrating SARS-CoV-2 nucleocapsid antibody detection in CHO cell culture fluid across the timepoints indicated using the DARQ assays of the present disclosure.

FIGS. 4A-4D: Representative results demonstrating SARS-CoV-2 nucleocapsid antibody detection in plasma samples across the timepoints indicated using the DARQ assays of the present disclosure.

### DETAILED DESCRIPTION

Accurate quantification of glycoproteins (e.g., mAbs) in complex media derived from industrial and patient samples, is vital to determine production efficiency or pharmacokinetic properties. To date, target glycoprotein quantification requires time and labor intensive assays. As described further herein, embodiments of the present disclosure include a novel bioassay termed Dual-Affinity Ratiometric Quenching (“DARQ”), which combines selective biorecognition and quenching of fluorescence signals for rapid and sensitive quantification of target glycoproteins (e.g., therapeutic monoclonal antibodies). This assay used the affinity complexation of a target glycoprotein by a protein binding moiety (e.g., PrL, which targets the Fab region of the antibody) and a glycan epitope binding moiety (e.g., a lectin or glycan-recognizing antibody), respectively labeled with fluorescein and rhodamine. Within the affinity complex, the glycoprotein acts as a scaffold framing the labeled affinity tags (PrL and lectin) in a molecular proximity that results in ratiometric quenching of their fluorescence emission. Notably, the altered (e.g., decrease) in fluorescence emission intensity is linearly dependent upon glycoprotein concentration in solution.

In accordance with this, embodiments of the present disclosure provide a rapid bioassay for the detection and rapid quantification of different glycoforms (i.e., a variant or isoform of a protein that differs only with respect to the number or type of attached glycan(s)) of a target protein either as purified or as present in a complex sample. As described herein, the bioassay of the present disclosure can be used to detect and quantify any glycoprotein-of-interest, provided that the glycoprotein-of-interest is capable of being simultaneously bound by a glycan epitope binding moiety or agent (e.g., lectin) and a protein binding moiety or agent (e.g., an antibody that recognizes a peptide epitope or a glycan epitope that is not recognized by the glycan epitope binding moiety or agent).

Generally, glycoproteins display either N-linked oligosaccharides (N-glycans) or O-Linked oligosaccharides (O-glycans). N-linked oligosaccharides are conjugated to asparagine or glutamine residues. O-Linked oligosaccharides are linked to serine, threonine, or tyrosine residues. The N-linked glycosylation is the most prevalent form of glycosylation and one of the most complex posttranslational modification. It determines both structural and functional characteristics of the protein. N-linked glycosylation includes three main patterns: High Mannose, Complex, and Hybrid, which share a common core structure including the first two N-acetylglucosamine residues and the first three mannose residues. The residues found in N-linked glycosylation include: N-Acetylglucosamine (GlcNAc), Mannose, Galactose, Xylose, Fucose, N-Acetylneuraminic acid, (Neu5Ac), and N-Glycolylneuraminic acid (Neu5Gc).

In one example, most therapeutic monoclonal antibodies (mAbs), the heavy chains contain an N-linked glycosylation site at asparagine 297 within the fragment crystallizable (Fc) region. In addition, some mAbs are also N-glycosylated within the fragment antigen-binding (Fab) region. The glycosylation pattern of the Fc region plays a key role in modulating effector function and pharmacokinetics. Specifically, the interaction of the Fc region with Fc-γ receptors (FcγR) influences immune effector functions including antibody-dependent cell-mediated cytotoxicity (ADCC). The absence of Fucose linked to the innermost GlcNAc (“core” fucose) enhances binding to Fcγ RIIIa and ADCC activity. Consequently, glycol-engineering approaches have been developed to generate a-fucosyl antibodies with enhanced ADCC activity. Therefore, most current therapeutic mAbs produced by CHO cells do not display a core fucose. Additionally, galactosylation can also enhance Fcγ RIIIa binding, and is additive to the ADCC effect of afucosylation. Terminal galactose also increases binding to complement 1q (C1q), leading to enhanced complement-dependent cytotoxicity (CDC). Therefore, most current therapeutic mAbs produced by CHO cells display a (terminal) galactose. Terminal sialic acid is anti-inflammatory, particularly when linked α(2,6) to galactose as opposed to the α(2,3) linkage produced by CHO cells. The presence of sialic acid residues may also reduce ADCC activity, and reduces clearance by shielding galactose from a sialo-glycoprotein receptors. Therefore, most current therapeutic mAbs produced by CHO cells display a terminal sialic acid. The O-linked glycosylation is less prevalent and relies on glycan conjugation to Serine or Threonine, although few instances of glucosylgalactose disaccharide conjugated to Hydroxylysine (Hyl) or an arabinose residue conjugated to Hydroxyproline (Hyp) have been reported. The first sugar residue is usually NAcetylgalactosamine (GalNAc); less commonly, galactose, mannose or xylose. However, other patterns have been reported, wherein the first residue is an O-Mannose, or O-Galactose, or O-Fucose, or O-Glucose. The residues found in O-linked glycosylation include N-Acetylgalactosamine, N-Acetylglucosamine, Mannose, Galactose. Xylose, N-Acetylneuraminic acid, (Neu5Ac), and Glucuronic acid. N-glycosylation of human antibodies has been identified in all IgG subclasses (IgG1, 2, 3, 4), IgA, IgM, IgD, and IgE. O-glycosylation of human antibodies has been identified in the hinge region of IgG3, IgA1 and IgD. Most therapeutic mAbs are IgG1 and IgG4. Therefore, for most therapeutic mAbs, N-glycosylation is the sole glycosylation.

Taken together, the “Glyco-DARQ” (Dual-Affinity Ratiometric Quenching) bioassay of the present disclosure combines selective biorecognition and quenching of fluorescence signals for rapid detection and sensitive quantification of N-glycosylation patterns on a glycoprotein-of-interest, such as therapeutic monoclonal antibodies (mAbs). In one example, the assay is based on the affinity complexation of the Fc region of a target mAb by Protein A or Protein G (i.e., PrA or PrG, which target the Fc region of the mAb, irrespective of its glycoform) and a lectin, namely a protein that specifically targets a glycan residue or a N-linked glycosylation pattern (therefore, different lectins can bind selectively only one, or few, or all glycoform(s) of the mAb). The PrA or PrG is labeled with a high-energy fluorophore (e.g., fluorescein). The lectin(s) is/are labeled with a low-energy fluorophore (e.g., rhodamine). Within the affinity complex, the mAb acts as a scaffold framing the labeled affinity tags (PrA/G and lectin) in a molecular proximity that results in ratiometric quenching of their fluorescence emission. In this exemplary bioassay, the decrease in fluorescence emission intensity is linearly dependent upon the concentration in solution of the mAb glycoform targeted by the adopted lectin.

Section headings as used in this section and the entire disclosure herein are merely for organizational purposes and are not intended to be limiting.

### 1. Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. The phrase “in one embodiment” as used herein does not necessarily refer to the same embodiment, though it may. Furthermore, the phrase “in another embodiment” as used herein does not necessarily refer to a different embodiment, although it may. Thus, as described below, various embodiments of the invention may be readily combined, without departing from the scope or spirit of the invention. All publications, patent applications, patents and other references mentioned herein are incorporated by reference in their entirety. The materials, methods, and examples disclosed herein are illustrative only and not intended to be limiting.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The singular forms “a,” “and” and “the” include plural references unless the context clearly dictates otherwise. The present disclosure also contemplates other embodiments “comprising.” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

“Correlated to” as used herein refers to compared to.

“Coefficient of variation” (CV), also known as “relative variability,” is equal to the standard deviation of a distribution divided by its mean.

“Component,” “components,” or “at least one component,” refer generally to an glycan epitope binding moiety, a peptide or protein binding moiety, a target glycoprotein, a fluorophore, a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that can be included in a kit for assay of a test sample in accordance with the methods described herein and other methods known in the art based on the present disclosure.

“Controls” as used herein generally refers to a reagent whose purpose is to evaluate the performance of a measurement system in order to assure that it continues to produce results within permissible boundaries (e.g., boundaries ranging from measures appropriate for a research use assay on one end to analytic boundaries established by quality specifications for a commercial assay on the other end). To accomplish this, a control should be indicative of patient results and optionally should somehow assess the impact of error on the measurement (e.g., error due to reagent stability, calibrator variability, instrument variability, and the like).

“Dynamic range” as used herein refers to range over which an assay readout is proportional to the amount of target molecule or analyte in the sample being analyzed. The dynamic range can be the range of linearity of the standard curve.

“Limit of Blank (LoB)” as used herein refers to the highest apparent analyte concentration expected to be found when replicates of a blank sample containing no analyte are tested.

“Limit of Detection (LoD)” as used herein refers to the lowest concentration of the measurand (i.e., a quantity intended to be measured) that can be detected at a specified level of confidence. The level of confidence is typically 95%, with a 5% likelihood of a false negative measurement. LoD is the lowest analyte concentration likely to be reliably distinguished from the LoB and at which detection is feasible. LoD can be determined by utilizing both the measured LoB and test replicates of a sample known to contain a low concentration of analyte. The LoD term used herein is based on the definition from Clinical and Laboratory Standards Institute (CLSI) protocol EP17-A2 (“Protocols for Determination of Limits of Detection and Limits of Quantitation; Approved Guideline—Second Edition,” EP17A2E, by James F. Pierson-Perry et al., Clinical and Laboratory Standards Institute, Jun. 1, 2012).

“Limit of Quantitation (LoQ)” as used herein refers to the lowest concentration at which the analyte can not only be reliably detected but at which some predefined goals for bias and imprecision are met. The LoQ may be equivalent to the LoD or it could be at a much higher concentration.

“Reference level” as used herein refers to an assay cutoff value that is used to assess diagnostic, prognostic, or therapeutic efficacy and that has been linked or is associated herein with various clinical parameters (e.g., presence of disease, stage of disease, severity of disease, progression, non-progression, or improvement of disease, etc.). It is well-known that reference levels may vary depending on the nature of the assay (e.g., antibodies employed, reaction conditions, sample purity, etc.) and that assays can be compared and standardized. Whereas the precise value of the reference level may vary between assays, the embodiments as described herein should be generally applicable and capable of being extrapolated to other assays.

“Antibody” and “antibodies” as used herein refers to monoclonal antibodies, monospecific antibodies (e.g., which can either be monoclonal, or may also be produced by other means than producing them from a common germ cell), multispecific antibodies, human antibodies, humanized antibodies (fully or partially humanized), animal antibodies such as, but not limited to, a bird (for example, a duck or a goose), a shark, a whale, and a mammal, including a non-primate (for example, a cow, a pig, a camel, a llama, a horse, a goat, a rabbit, a sheep, a hamster, a guinea pig, a cat, a dog, a rat, a mouse, etc.) or a non-human primate (for example, a monkey, a chimpanzee, etc.), recombinant antibodies, chimeric antibodies, single-chain Fvs (“scFv”), single chain antibodies, single domain antibodies, Fab fragments, F(ab′) fragments, F(ab′)₂ fragments, disulfide-linked Fvs (“sdFv”), and anti-idiotypic (“anti-Id”) antibodies, dual-domain antibodies, dual variable domain (DVD) or triple variable domain (TVD) antibodies (dual-variable domain immunoglobulins and methods for making them are described in Wu, C., et al., Nature Biotechnology, 25(11):1290-1297 (2007) and PCT International Application WO 2001/058956, the contents of each of which are herein incorporated by reference), or domain antibodies (dAbs) (e.g., such as described in Holt et al. (2014) Trends in Biotechnology 21:484-490), and including single domain antibodies sdAbs that are naturally occurring, e.g., as in cartilaginous fishes and camelid, or which are synthetic, e.g., nanobodies, VHH, or other domain structure), and functionally active epitope-binding fragments of any of the above. In particular, antibodies include immunoglobulin molecules and immunologically active fragments of immunoglobulin molecules, namely, molecules that contain an analyte-binding site. Immunoglobulin molecules can be of any type (for example, IgG, IgE, IgM, IgD, IgA, and IgY), class (for example, IgG1, IgG2, IgG3, IgG4, IgA1, and IgA2).

“Epitope,” or “epitopes,” or “epitopes of interest” refer to a site(s) on any molecule that is recognized and can bind to a complementary site(s) on its specific binding partner. The molecule and specific binding partner are part of a specific binding pair. For example, an epitope can be on a polypeptide, a protein, a hapten, a carbohydrate antigen (such as, but not limited to, glycolipids, glycoproteins or lipopolysaccharides), or a polysaccharide. Its specific binding partner can be, but is not limited to, an antibody.

The terms “specific binding partner,” “specific binding member,” and “binding member” are used interchangeably herein and refer to one of two or more different molecules that specifically recognize the other molecule compared to substantially less recognition of other molecules.

As used herein, when an antibody or other entity (e.g., antigen binding domain) “specifically recognizes” or “specifically binds” an antigen or epitope, it preferentially recognizes the antigen in a complex mixture of proteins and/or macromolecules, and binds the antigen or epitope with affinity which is substantially higher than to other entities not displaying the antigen or epitope. In this regard, “affinity which is substantially higher” means affinity that is high enough to enable selective detection of a target antigen or epitope which is distinguished from entities using a desired assay or measurement apparatus. Typically, it means binding affinity having a binding constant (Kₐ) of at least 10⁷ M⁻¹ (e.g., >10⁷ M⁻¹, >10⁸ M⁻¹, >10⁹ M⁻¹, >10¹⁰ M⁻¹, >10¹¹>M⁻¹, >10¹² M⁻¹, >10¹³>M⁻¹, etc.). In certain such embodiments, an antibody is capable of binding different antigens so long as the different antigens comprise that particular epitope. In certain instances, for example, homologous proteins from different species may comprise the same epitope.

As used herein, the term “subject” refers to any human or animal (e.g., non-human primate, rodent, feline, canine, bovine, porcine, equine, caprine, etc.).

As used herein, the term “sample” is used in its broadest sense and encompass materials obtained from any source. As used herein, the term “sample” is used to refer to materials obtained from a biological source, for example, obtained from animals (including humans), and encompasses any fluids, solids, and tissues. In some embodiments, the sample is a whole blood sample, a plasma sample, a serum sample, a urine sample, a saliva sample, or a tissue sample. However, these examples are not to be construed as limiting the types of samples that find use with the present disclosure. In some embodiments, the sample is a plant sample, a soil sample, a water sample, a cell culture sample, a cell lysate sample, or a cell culture media sample.

As used herein, the term “antibody sample” refers to an antibody-containing composition (e.g., plasma, blood, purified antibodies, blood or plasma fractions, blood or plasma components etc.) taken from or provided by a donor (e.g., natural source) or obtained from a synthetic, recombinant, other in vitro source, or from a commercial source. The antibody sample may exhibit elevated titer of a particular antibody or set of antibodies based on the pathogenic/antigenic exposures (e.g., natural exposure or through vaccination) of the donor or the antibodies engineered to be produced in the synthetic, recombinant, or in vitro context.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. Dual-Affinity Ratiometric Quenching (DARQ) Bioassay

The current technologies utilized by analytical labs at both clinical and biomanufacturing sites for glycoprotein quantification in complex media are laborious and involve expensive consumables or equipment. To overcome these issues, a novel “DARQ” (dual affinity ratiometric quenching) assay was developed for glycoprotein quantification that is rapid, robust, and reproducible, and accurate. The assay relies on a highly selective affinity interaction between a target glycoprotein, a labeled glycan epitope biding moiety (e.g., lectin), and a labeled peptide epitope binding moiety (e.g., Protein L). The glycoprotein captures the fluorescently labeled proteins, framing them in a supramolecular affinity complex, wherein the fluorophore labels are constrained in proximity within a dense proteinaceous structure. This translates into an alteration of their fluorescent emission (e.g., increase or decrease) and/or their fluorescent emission lifetime that is linearly dependent upon the glycoprotein concentration. Control assays performed using different combinations of fluorescent affinity tags, while not revealing the specific nature of the mechanism at hand, indicate that it is of the nature of energy transfer quenching. In one embodiment, the high binding strength and selectivity of both a lectin and Protein L to the target glycoprotein accelerates the formation of the affinity complex, leaving no residual free glycoprotein in solution. The combination of biorecognition and fluorescence quenching makes the assay rapid (˜5 min), highly sensitive (<0.5 ng·mL⁻¹), and reproducible (CoV<1.7%). Reliance on a protein binding moiety that recognizes an epitope that is different from (or does not interfere with) the glycan epitope recognized by the lectin (e.g., Protein L, which targets the antibody's Fab region of κI, κIII, and κIV subtypes), does not limit the applicability of the assay, given for example, that most therapeutic antibodies currently on the market belong to the κI group. This makes DARQ an ideal mix-and-read assay for at-line monitoring of antibody concentration in bioprocessing fluids (e.g., clarified harvest and chromatographic fractions) or a point-of-care test (POCT) for clinical laboratories.

Embodiments of the present disclosure include a ratiometric bioassay method for detecting a target glycoprotein in a sample. In accordance with these embodiments, the method includes combining a glycan epitope binding moiety such as a lectin coupled to a first fluorophore, a protein binding moiety coupled to a second fluorophore, and a sample comprising or suspected of comprising a target glycoprotein. The lectin coupled to the first fluorophore, the protein binding moiety coupled to the second fluorophore, and the sample can be combined in any suitable vessel, container, tube, and the like that allows for a binding complex to form. The method includes exposing the lectin coupled to the first fluorophore, the protein binding moiety coupled to the second fluorophore, and the sample to light (e.g., fluorescent light) comprising an excitation wavelength of the first and/or the second fluorophore, and detecting emission from the first and/or the second fluorophore. In some embodiments, the emission from the first and the second fluorophore is decreased, and wherein the decreased emission is proportional to the glycoprotein concentration in the sample. In some embodiments, the emission from the first and the second fluorophore is increased, and wherein the increased emission is proportional to the glycoprotein concentration in the sample. In some embodiments, the emission lifetime from the first and the second fluorophore is altered, and wherein the altered emission lifetime is proportional to the glycoprotein concentration in the sample. In some embodiments, the ratiometric bioassays of the present disclosure involve fluorescence quenching of the first and/or the second fluorophore to determine antibody concentration, rather than a FRET-based mechanism. That is, the ratiometric bioassays of the present disclosure do not rely generally on emission from a first fluorophore to excite a second fluorophore.

In some embodiments, the lectin coupled to the first fluorophore is any lectin that is capable of binding a glycan epitope on the target glycoprotein in the sample. The lectin can be the fully characterized lectin peptide sequence identified as being capable of binding to the target glycoprotein, or it can be any fragment or derivative thereof, provided the portion of the lectin that binds the glycan epitope on the target glycoprotein is functionally intact. As would be readily apparent to one of ordinary skill in the art based on the present disclosure, the compositions and methods provided herein can include the use of any lectin known to bind a target glycoprotein, and any lectin subsequently developed or identified as being capable of binding a target glycoprotein. Lectins can be obtained through any means known in the art, including but not limited to, chemical synthesis, protein purification, and genetic and cellular engineering. In some embodiments, lectin includes, but is not limited to, LTL (Lotus tetragonolobus), PSA (Pisum sativum), LCA (Lens culinaris), UEA-I (Ulex europaeus), AOL (Aspergillus oryzae), AAL (Aleuria aurantia), MAL_I (Maackia amurensis), SNA (Sambucus nigra), SSA (Sambucus sieboldiana), TJA-I (Trichosanthes japonica), PHAL (Phaseolus vulgaris), ECA (Erythrina cristagalli), RCA120 (Ricinus communis), PHAE (Phaseolus vulgaris), DSA (Datura stramonium), GSL-II (Griffonia simplicifolia), NPA (Narcissus pseudonarcissus), ConA (Canavalia ensiformis), GNA (Galanthus nivalis), HHL (Hippeastrum hybrid), ACG (mushroom. Agrocybe cylindracea), TxLCI (Tulipa gesneriana), BPL (Bauhinia purpurea), TJA-II (Tanthes japonica). EEL (Euonymus europaeus), ABA (fungus, Agaricus bisporus), LEL (tomato, Lycopersicon esculentum), STL (potato, Solanum tuberosum), UDA (Urtica dioica), PWM (pokeweed, Phytolacca Americana), Jacalin (Artocarpus integrifolia), PNA (peanut, Arachis hypogaea), WFA (Wisteria floribunda), ACA (Amaranthus caudatus), MPA (Maclura pomifera), HPA (snail, Helix pomatia), VVA (Vicia villosa), DBA (Dolichos biflorus), SBA (soybean, Dolichos biflorus), Calsepa (Calystegia sepium), PTL-I (Psophocarpus tetragonolobus), MAH (Maackia amurensis), WGA (wheat germ, Triticum aestivum), GSL-I A4 (Griffonia simplicifolia), and GSL-I B4 (Griffonia simplicifolia), or a glycan epitope binding fragment thereof.

As described further herein, the lectin can be coupled to a first fluorophore that can be either a high-energy fluorophore or a low-energy fluorophore. In some embodiments, the lectin can be coupled to another agent in addition to the first fluorophore, provided that the additional agent does not interfere with the ability of the fluorophore to emit fluorescence or the ability of the lectin to bind the target glycoprotein. As would be recognized by one of ordinary skill in the art based on the present disclosure, coupling a fluorophore to a lectin can be done by any means known in the art. In some embodiments, coupling a lectin to a fluorophore is referred to as functional coupling because both the fluorophore and the lectin maintain at least some degree of functionality as compared to their functionality prior to coupling.

In some embodiments, the lectin is coupled to the first fluorophore at a lectin-to-fluorophore ratio of about 1:0.2 to about 1:20. In some embodiments, the lectin is coupled to the first fluorophore at a lectin-to-fluorophore ratio of about 1:0.5 to about 1:20. In some embodiments, the lectin is coupled to the first fluorophore at a lectin-to-fluorophore ratio of about 1:1 to about 1:20. In some embodiments, the lectin is coupled to the first fluorophore at a lectin-to-fluorophore ratio of about 1:1.5 to about 1:20. In some embodiments, the lectin is coupled to the first fluorophore at a lectin-to-fluorophore ratio of about 1:2 to about 1:20. In some embodiments, the lectin is coupled to the first fluorophore at a lectin-to-fluorophore ratio of about 1:3 to about 1:20. In some embodiments, the lectin is coupled to the first fluorophore at a lectin-to-fluorophore ratio of about 1:4 to about 1:20. In some embodiments, the lectin is coupled to the first fluorophore at a lectin-to-fluorophore ratio of about 1:5 to about 1:20. In some embodiments, the lectin is coupled to the first fluorophore at a lectin-to-fluorophore ratio of about 1:10 to about 1:20. In some embodiments, the lectin is coupled to the first fluorophore at a lectin-to-fluorophore ratio of about 1:15 to about 1:20. In some embodiments, the lectin is coupled to the first fluorophore at a lectin-to-fluorophore ratio of about 1:2.0 to about 1:20. In some embodiments, the lectin is coupled to the first fluorophore at a lectin-to-fluorophore ratio of about 1:0.2 to about 1:15. In some embodiments, the lectin is coupled to the first fluorophore at a lectin-to-fluorophore ratio of about 1:0.2 to about 1:10. In some embodiments, the lectin is coupled to the first fluorophore at a lectin-to-fluorophore ratio of about 1:0.2 to about 1:5. In some embodiments, the lectin is coupled to the first fluorophore at a lectin-to-fluorophore ratio of about 1:0.2 to about 1:4. In some embodiments, the lectin is coupled to the first fluorophore at a lectin-to-fluorophore ratio of about 1:0.2 to about 1:3. In some embodiments, the lectin is coupled to the first fluorophore at a lectin-to-fluorophore ratio of about 1:0.2 to about 1:2. In some embodiments, the lectin is coupled to the first fluorophore at a lectin-to-fluorophore ratio of about 1:0.2 to about 1:1. In some embodiments, the lectin is coupled to the first fluorophore at a lectin-to-fluorophore ratio of about 1:1 to about 1:10. In some embodiments, the lectin is coupled to the first fluorophore at a lectin-to-fluorophore ratio of about 1:2 to about 1:8. In some embodiments, the lectin is coupled to the first fluorophore at a lectin-to-fluorophore ratio of about 1:3 to about 1:6.

In some embodiments, the protein binding moiety comprises a polypeptide capable of binding a region of the target glycoprotein that does not comprise the glycan epitope capable of being bound by the lectin. In some embodiments, the protein binding moiety coupled to the second fluorophore includes any protein binding moiety that is capable of binding a region of the target glycoprotein that does not comprise the glycan epitope. The protein binding moiety can be a fully characterized polypeptide or protein identified as being capable of binding to the target glycoprotein, or it can be any fragment or derivative thereof, provided the portion of the protein binding moiety that can bind to the target glycoprotein is functionally intact. As would be recognized by one of ordinary skill in the art based on the present disclosure, the compositions and methods provided herein can include the use of any protein binding moiety known to bind a target glycoprotein, and any protein binding moiety subsequently developed or identified as being capable of binding a target glycoprotein. For example, in some embodiments, the protein binding moiety comprises Protein L, Protein A, or Protein G, including any fragments, variants, or derivatives thereof.

As described further herein, the protein binding moiety is coupled to a second fluorophore that can be either a high-energy fluorophore or a low-energy fluorophore. In some embodiments, the protein binding moiety can be coupled to another agent in addition to the second fluorophore, provided that the additional agent does not interfere with the ability of the fluorophore to emit fluorescence or the ability of the protein binding moiety to bind the target glycoprotein. As would be recognized by one of ordinary skill in the art based on the present disclosure, coupling a fluorophore to a protein binding moiety can be done by any means known in the art. In some embodiments, coupling a protein binding moiety to a fluorophore is referred to as functional coupling because both the fluorophore and the protein binding moiety maintain at least some degree of functionality as compared to their functionality prior to coupling.

In some embodiments, the protein binding moiety is coupled to the second fluorophore at a moiety-to-fluorophore ratio of about 1:0.2 to about 1:20. In some embodiments, the protein binding moiety is coupled to the second fluorophore at a moiety-to-fluorophore ratio of about 1:0.5 to about 1:20. In some embodiments, the protein binding moiety is coupled to the second fluorophore at a moiety-to-fluorophore ratio of about 1:1 to about 1:20. In some embodiments, the protein binding moiety is coupled to the second fluorophore at a moiety-to-fluorophore ratio of about 1:1.5 to about 1:20. In some embodiments, the protein binding moiety is coupled to the second fluorophore at a moiety-to-fluorophore ratio of about 1:2 to about 1:20. In some embodiments, the protein binding moiety is coupled to the second fluorophore at a moiety-to-fluorophore ratio of about 1:3 to about 1:20. In some embodiments, the protein binding moiety is coupled to the second fluorophore at a moiety-to-fluorophore ratio of about 1:4 to about 1:20. In some embodiments, the protein binding moiety is coupled to the second fluorophore at a moiety-to-fluorophore ratio of about 1:5 to about 1:20. In some embodiments, the protein binding moiety is coupled to the second fluorophore at a moiety-to-fluorophore ratio of about 1:10 to about 1:20. In some embodiments, the protein binding moiety is coupled to the second fluorophore at a moiety-to-fluorophore ratio of about 1:15 to about 1:20. In some embodiments, the protein binding moiety is coupled to the second fluorophore at a moiety-to-fluorophore ratio of about 1:2.0 to about 1:20. In some embodiments, the protein binding moiety is coupled to the second fluorophore at a moiety-to-fluorophore ratio of about 1:0.2 to about 1:15. In some embodiments, the protein binding moiety is coupled to the second fluorophore at a moiety-to-fluorophore ratio of about 1:0.2 to about 1:10. In some embodiments, the protein binding moiety is coupled to the second fluorophore at a moiety-to-fluorophore ratio of about 1:0.2 to about 1:5. In some embodiments, the protein binding moiety is coupled to the second fluorophore at a moiety-to-fluorophore ratio of about 1:0.2 to about 1:4. In some embodiments, the protein binding moiety is coupled to the second fluorophore at a moiety-to-fluorophore ratio of about 1:0.2 to about 1:3. In some embodiments, the protein binding moiety is coupled to the second fluorophore at a moiety-to-fluorophore ratio of about 1:0.2 to about 1:2. In some embodiments, the protein binding moiety is coupled to the second fluorophore at a moiety-to-fluorophore ratio of about 1:0.2 to about 1:1. In some embodiments, the protein binding moiety is coupled to the second fluorophore at a moiety-to-fluorophore ratio of about 1:1 to about 1:10. In some embodiments, the protein binding moiety is coupled to the second fluorophore at a moiety-to-fluorophore ratio of about 1:2 to about 1:8. In some embodiments, the protein binding moiety is coupled to the second fluorophore at a moiety-to-fluorophore ratio of about 1:3 to about 1:6.

As described further herein, the bioassay methods of the present disclosure include exposing the lectin coupled to the first fluorophore, the protein binding moiety coupled to the second fluorophore, and the sample to fluorescent light comprising an excitation wavelength of the first and/or the second fluorophore, and detecting fluorescence emission from the first and/or the second fluorophore. In accordance with these embodiments, the first and second fluorophores can be either high-energy or low-energy fluorophores. In some embodiments, the lectin is coupled to a first fluorophore that is a high-energy fluorophore, and the protein binding moiety is coupled to a second fluorophore that is a low-energy fluorophore. In some embodiments, the lectin is coupled to a first fluorophore that is a low-energy fluorophore, and the protein binding moiety is coupled to a second fluorophore that is a high-energy fluorophore. As described further herein, a high-energy fluorophore (or higher-energy fluorophore) generally has an excitation spectra bathocrhomically shifted from the excitation spectra of a low-energy fluorophore (or lower-energy fluorophore). In addition, the high-energy fluorophore may have an emission spectra that is also bathocrhomically shifted from the excitation spectra of the low-energy fluorophore.

In some embodiments, the high-energy fluorophore and/or the low-energy fluorophore include any fluorophore(s) capable of binding N-hydroxysuccinimde (NHS). In some embodiments, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532. In some embodiments, the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750. In some embodiments, the lectin is coupled to fluorescein or Alexafluor 350. In some embodiments, the protein binding moiety is coupled to Rhodamine.

In some embodiments, the method further comprises determining the concentration of the target glycoprotein in the sample based on the altered emission of the first and the second fluorophore. In some embodiments, the altered emission comprises decreased emission, increased emission, and/or a change in emission lifetime. In some embodiments, the method further includes determining the concentration of a target glycoprotein in a sample based on the increase or decrease in fluorescence emission of the first and/or the second fluorophore. In some embodiments, the concentration of a target glycoprotein in a sample is based on the increase or decrease in fluorescence emission of the first fluorophore. In some embodiments, the concentration of a target glycoprotein in a sample is based on the increase or decrease in fluorescence emission of the second fluorophore. In some embodiments, the concentration of a target glycoprotein in a sample is based on the increase or decrease in fluorescence emission of the first fluorophore and the second fluorophore.

Generally, fluorescence lifetime (FLT) is the time a fluorophore spends in the excited state before emitting a photon and returning to the ground state. FLT can vary from picoseconds to hundreds of nanoseconds depending on the fluorophore. In some embodiments, the method further includes determining the concentration of a target glycoprotein in a sample based on the increase or decrease in FLT of the first and/or the second fluorophore. In some embodiments, the concentration of a target glycoprotein in a sample is based on the increase or decrease in FLT of the first fluorophore. In some embodiments, the concentration of a target glycoprotein in a sample is based on the increase or decrease in FLT of the second fluorophore. In some embodiments, the concentration of a target glycoprotein in a sample is based on the increase or decrease in FLT of the first fluorophore and the second fluorophore.

In some embodiments, the first fluorophore and the second fluorophore are present in the composition at a first-to-second fluorophore ratio of about 1:1 to about 1:20. In some embodiments, the first fluorophore and the second fluorophore are present in the composition at a first-to-second fluorophore ratio of about 1:1 to about 1:15. In some embodiments, the first fluorophore and the second fluorophore are present in the composition at a first-to-second fluorophore ratio of about 1:1 to about 1:10. In some embodiments, the first fluorophore and the second fluorophore are present in the composition at a first-to-second fluorophore ratio of about 1:1 to about 1:5. In some embodiments, the first fluorophore and the second fluorophore are present in the composition at a first-to-second fluorophore ratio of about 1:5 to about 1:20. In some embodiments, the first fluorophore and the second fluorophore are present in the composition at a first-to-second fluorophore ratio of about 1:10 to about 1:20. In some embodiments, the first fluorophore and the second fluorophore are present in the composition at a first-to-second fluorophore ratio of about 1:15 to about 1:20. In some embodiments, the first fluorophore and the second fluorophore are present in the composition at a first-to-second fluorophore ratio of about 1:5 to about 1:15. In some embodiments, the first fluorophore and the second fluorophore are present in the composition at a first-to-second fluorophore ratio of about 1:5 to about 1:10. In some embodiments, the first fluorophore and the second fluorophore are present in the composition at a first-to-second fluorophore ratio of about 1:10 to about 1:20. In some embodiments, the first fluorophore and the second fluorophore are present in the composition at a first-to-second fluorophore ratio of about 1:10 to about 1:15.

In some embodiments, the method further includes incubating the sample comprising or suspected of comprising the target glycoprotein, the lectin coupled to the first fluorophore, and the protein binding moiety coupled to the second fluorophore for 30 minutes or less prior to measuring the fluorescent emission of the first and/or the second fluorophore. In some embodiments, the method further includes incubating the sample comprising or suspected of comprising the target glycoprotein, the lectin coupled to the first fluorophore, and the protein binding moiety coupled to the second fluorophore for 20 minutes or less prior to measuring the fluorescent emission of the first and/or the second fluorophore. In some embodiments, the method further includes incubating the sample comprising or suspected of comprising the target glycoprotein, the lectin coupled to the first fluorophore, and the protein binding moiety coupled to the second fluorophore for 10 minutes or less prior to measuring the fluorescent emission of the first and/or the second fluorophore. In some embodiments, the method includes incubating for about 1 minute, about 2 minutes, about 3 minutes, about 4 minutes, about 5 minutes, about 6 minutes, about 7 minutes, about 8 minutes, about 9 minutes, about 10 minutes, about 11 minutes, about 12 minutes, about 13 minutes, about 14 minutes, about 15 minutes, about 16 minutes, about 17 minutes, about 18 minutes, about 19 minutes, about 20 minutes, about 21 minutes, about 22 minutes, about 23 minutes, about 24 minutes, about 25 minutes, about 26 minutes, about 27 minutes, about 28 minutes, about 29 minutes, or about 30 minutes. In some embodiments, the method includes incubating for over 30 minutes.

In accordance with the above embodiments, the bioassay methods and compositions of the present disclosure can be used with any sample that contains, or is suspected of containing, a target glycoprotein. In some embodiments, sample is undiluted (e.g., sampled directly) prior to the bioassay being performed. In some embodiments, sample is diluted prior to the bioassay being performed, such as with a suitable buffer or other agent. In some embodiments, the sample is obtained from an organism or a portion of an organism. In some embodiments, the sample is obtained from a bodily fluid of a mammal (e.g., a human). In some embodiments, the sample is a whole blood sample, a plasma sample, a scrum sample, a urine sample, a saliva sample, or a tissue sample. In some embodiments, the sample is obtained from an industrial biological, chemical, or biochemical process. For example, in some embodiments, the sample is a cell culture sample, a cell lysate sample, or a cell culture media sample. As would be recognized by one of ordinary skill in the art based on the present disclosure, other samples may also be used with the bioassays of the present disclosure.

As described herein, the bioassay of the present disclosure can be used to detect and quantify any glycoprotein-of-interest, provided that the glycoprotein-of-interest is capable of being simultaneously bound by a glycan epitope binding moiety or agent (e.g., lectin) and a protein binding moiety or agent (e.g., an antibody that recognizes a peptide epitope or a glycan epitope that is not recognized by the glycan epitope binding moiety or agent). In some embodiments, the target glycoprotein is an antibody, a blood protein, a mammalian cell surface protein, or a growth factor. In some embodiments, the target glycoprotein is from a bacteria, virus, or fungus.

In accordance with the above embodiments, the present disclosure also provides compositions and methods for detecting a target antibody using the antigen to which it binds. Thus, embodiments of the present disclosure include a ratiometric bioassay method for detecting a target antibody in a sample. In some embodiments, the method includes combining an antigen, or fragment thereof, coupled to a first fluorophore and an antibody binding moiety coupled to a second fluorophore with a sample comprising or suspected of comprising a target antibody; exposing the antigen, or fragment thereof, coupled to the first fluorophore, the antibody binding moiety coupled to the second fluorophore, and the sample to light comprising an excitation wavelength of the first and/or the second fluorophore; and detecting emission from the first and/or the second fluorophore. In some embodiments, the emission from the first and the second fluorophore is altered, and wherein the altered emission is proportional to the antibody concentration in the sample.

In some embodiments, the target antibody is capable of binding the antigen. In some embodiments, the antibody binding moiety is capable of binding a region of the target antibody that does not interfere with the antigen binding. In some embodiments, the antibody binding moiety comprises Protein L, Protein A, Protein G or an antibody-binding fragment thereof.

In some embodiments, the first fluorophore is a high-energy fluorophore and the second fluorophore is a low-energy fluorophore. In some embodiments, the second fluorophore is a high-energy fluorophore and the first fluorophore is a low-energy fluorophore.

In some embodiments, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

In some embodiments, the low-energy fluorophore is selected from the group consisting of Rhodamine, Rhodamine B. Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750. In some embodiments, the antibody binding moiety is coupled to Rhodamine. In some embodiments, the antigen is coupled to fluorescein.

In some embodiments, the method further comprises determining the concentration of the target antibody in the sample based on the altered emission of the first and the second fluorophore. In some embodiments, the altered emission comprises decreased emission, increased emission, and/or a change in emission lifetime.

In some embodiments, the method further comprises incubating the sample comprising or suspected of comprising the target antibody, the antigen coupled to the first fluorophore, and the antibody binding moiety coupled to the second fluorophore for 30 minutes or less prior to measuring the emission of the first and/or the second fluorophore.

In some embodiments, the antigen is coupled to the first fluorophore at an antigen-to-fluorophore ratio of about 1:0.2 to about 1:20. In some embodiments, the antibody binding moiety is coupled to the second fluorophore at a moiety-to-fluorophore ratio of about 1:0.2 to about 1:20. In some embodiments, the first fluorophore and the second fluorophore are present in the composition at a first-to-second fluorophore ratio of about 1:1 to about 1:20.

In some embodiments, the sample is undiluted. In some embodiments, the sample is a whole blood sample, a plasma sample, a serum sample, a urine sample, a saliva sample, or a tissue sample. In some embodiments, sample is a plant sample, a soil sample, a water sample, a cell culture sample, a cell lysate sample, or a cell culture media sample. In some embodiments, the antigen is from a pathogenic organism. In some embodiments, the pathogenic organism is a bacteria, a virus, or a fungus. In some embodiments, the antigen is from SARS-CoV-2.

In some embodiments, the ratiometric bioassay methods and compositions of the present disclosure can be used to detect antibodies in a sample from a patient (e.g., whole blood sample, a plasma sample, a serum sample, a urine sample, a saliva sample, a tissue sample, and the like) that has been infected with, or is suspected of being infected with, a coronavirus (e.g., coronavirus OC43, coronavirus 229E, coronavirus NL63, coronavirus HKU1, MERS-CoV, SARS-CoV, or SARS-CoV-2 (COVID-19)). Currently, most available serological tests that detect SARS-CoV-2 antibodies are lateral flow assays (LFA) that are based on simple positive or negative detection of antibodies, which is feasible for inexpensive and point-of-care (POC) use and large-scale surveillance but not informative regarding the amount, type, or function of the antibodies. An alternative for accurately detecting antibodies against SARS-CoV-2 is the enzyme-linked immunosorbent assay (ELISA), which can measure not only the presence but also the titer (amount) and type (IgG, IgM, monomeric or dimeric IgA) of antibody. ELISA assays allow for a better measure of the strength of the humoral response, but are complex and can only be performed in a laboratory setting. Additionally, ELISA is not ideal for virus neutralization/blocking tests, which is crucially important in studying the humoral response during vaccine development and vaccination but not widely available. Current neutralization assays usually involve propagation of viruses and require such assays to be conducted in a biosafety level 3 (BSL3) lab settings, which unfortunately is unavailable to many researchers or the public.

Further, in accordance with the ratiometric bioassay methods and compositions described herein, embodiments of the present disclosure include detecting and/or quantifying coronavirus-neutralizing monoclonal antibodies in a sample obtained from a bioreactor wherein cells recombinantly engineered to express that monoclonal antibody have been cultured and produced said antibody, either as an intracellular or extracellular product. Currently, analytical assays utilized in the biopharmaceutical and biomanufacturing industries for the quantification of recombinant monoclonal antibodies rely on ELISA tests. The slow kinetics of ELISAs make them inherently off-line assays, thereby reducing the ability of optimizing the process in real time.

Neutralizing antibodies identified using the disclosed methods and compositions can specifically bind to any known or as yet undiscovered coronavirus, such as, for example, coronavirus OC43, coronavirus 229E, coronavirus NL63, coronavirus HKU1, MERS-CoV, SARS-CoV, or SARS-CoV-2 (COVID-19). In some embodiments, the neutralizing antibodies are directed against SARS-CoV-2 (COVID-19). In the context of the present disclosure a “neutralizing antibody” is an antibody that binds to a virus (e.g., a coronavirus) and interferes with the virus' ability to infect a host cell. Coronavirus spike proteins are known to elicit potent neutralizing-antibody and T-cell responses. The ability of a virus (e.g., coronavirus OC43, coronavirus 229E, coronavirus NL63, coronavirus HKU1, MERS-CoV, SARS-CoV, or SARS-CoV-2 (COVID-19)) to gain entry into cells and establish infection is mediated by the interactions of its Spike glycoproteins with human cell surface receptors. In the case of coronaviruses, Spike proteins are large type I transmembrane protein trimers that protrude from the surface of coronavirus virions. Each Spike protein comprises a large ectodomain (comprising S1 and S2), a transmembrane anchor, and a short intracellular tail. The S1 subunit of the ectodomain mediates binding of the virion to host cell-surface receptors through its receptor-binding domain (RBD). The S2 subunit fuses with both host and viral membranes, by undergoing structural changes.

As described further herein, the compositions and methods of the present disclosure can also be used to detect antibodies to SARS-CoV-2 nucleocapsid (N) proteins. Neutralizing antibodies directed against coronaviruses have been identified and isolated (see, e.g., Liu et al., Potent neutralizing antibodies directed to multiple epitopes on SARS-CoV-2 spike. Nature (2020). doi.org/10.1038/s41586-020-2571-7; Rogers et al., Science 15 Jun. 2020:eabc7520; DOI: 10.1126/science.abc7520; Alsoussi et al., J Immunol Jun. 26, 2020, ji2000583; DOI:/doi.org/10.4049/jimmunol.2000583; Kreer et al., Cell, S0092-8674(20)30821-7. 13 Jul. 2020, doi:10.1016/j.cell.2020.06.044; Tai et al., J Virol. 2017 Jan. 1; 91(1): e01651-16; and Niu et al., J Infect Dis. 2018 Oct. 15; 218(8): 1249-1260).

In accordance with the above, the ratiometric bioassay methods and compositions described herein can include the use of a coronavirus antigen, such as an antigen from the N protein or the RBD of the Spike protein of SARS-CoV-2, in combination with an antibody-binding moiety (e.g., Protein L, Protein A, or Protein G). In some embodiments, the coronavirus antigen is coupled to a first fluorophore, and the antibody-binding moiety is coupled to a second fluorophore, wherein the fluorescence emission from the first and the second fluorophore is reduced, and wherein the reduced fluorescence emission is proportional to the antibody concentration in the sample, as described further herein. In this manner, neutralizing antibodies directed against SARS-CoV-2 can be detected and/or quantified in a sample from a patient or from a recombinant source. In some embodiments, the coronavirus antigen may be prepared using routine molecular biology techniques, as would be recognized by one of ordinary skill in the art based on the present disclosure.

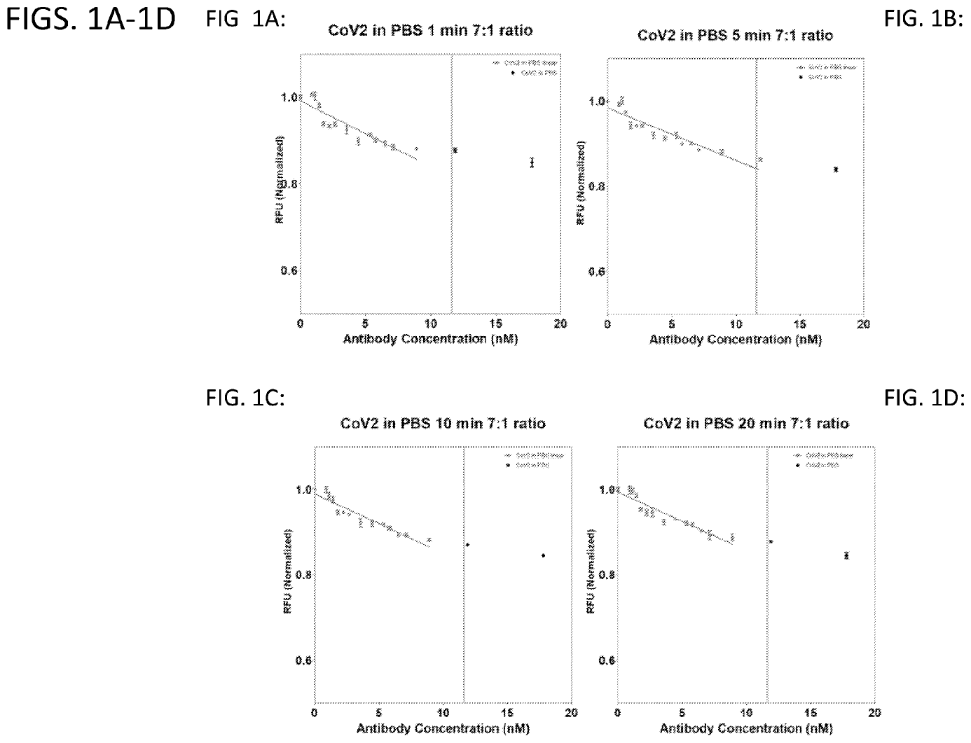

In accordance with these embodiments, experiments were conducted to assess the ability of the DARQ assays described herein to detect antibodies against SARS-CoV-2 nucleocapsid (N) protein (FIGS. 1-4). In particular, aliquots of 230 μL of ^{Rh}PrL/^{FL}CoV2 stock solution in PBS were dispensed in 48 wells of a 96-well nonbinding microplate, to which 20 μL aliquots of Anti-CoV2 solutions at different concentrations were added to achieve a final concentration in each well of 23.2 nM for both ^{Rh}PrL and ^{Fl}COV2 and varying between 0.89 and 18.4 nM for Anti-CoV2. Upon mixing, the samples analyzed by fluorescence spectrophotometry at the excitation wavelength of 480 nm and emission wavelengths of 525 and 573 nm, along with an excitation of 525 nm and emission at 573 nm. Aliquots of anti-CoV2 were made in PBS, saliva, CHO fluid, and plasma and the experiment was replicated as described above.

The results provided in FIGS. 1A-1D demonstrate SARS-CoV-2 nucleocapsid antibody detection in PBS across the timepoints indicated using the DARQ assays of the present disclosure. Aliquots of 230 μL of ^{Rh}PrL/^{FL}CoV2 stock solution in PBS to achieve a final concentration in each well of 23.2 nM for both ^{Rh}PrL and ^{Fl}COV2. Aliquots of 20 μL Anti-CoV2 solutions were added to achieve varying concentrations in well between 0.89 and 18.4 nM.

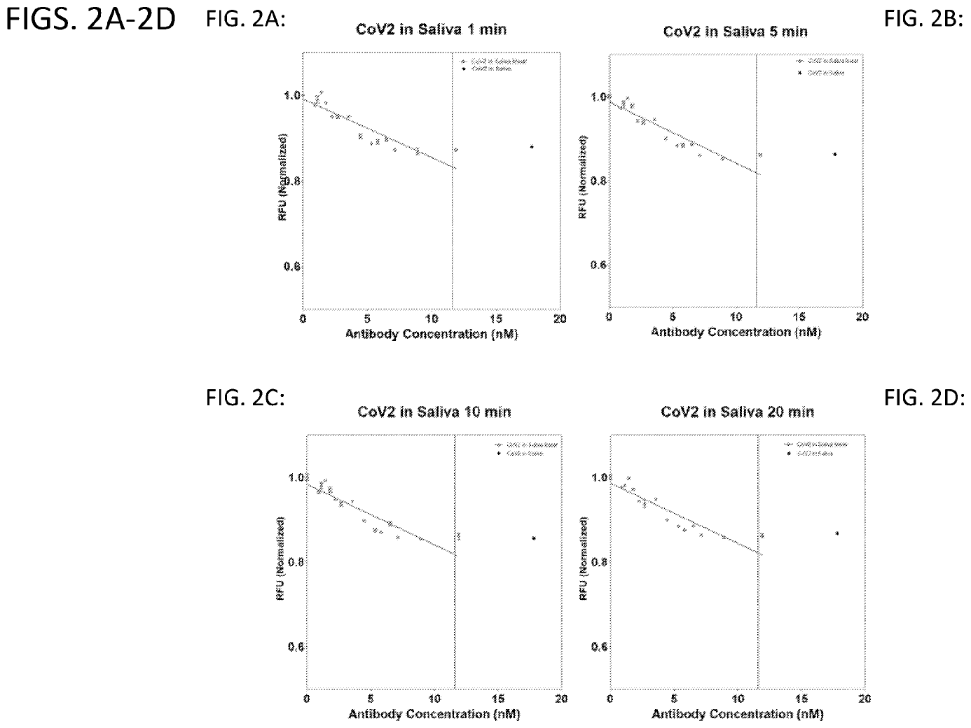

The results provided in FIGS. 2A-2D demonstrate SARS-CoV-2 nucleocapsid antibody detection in saliva samples across the timepoints indicated using the DARQ assays of the present disclosure. Aliquots of 230 μL of ^{Rh}PrL/^{FL}CoV2 stock solution in saliva to achieve a final concentration in each well of 23.2 nM for both ^{Rh}PrL and ^{Fl}COV2. Aliquots of 20 μL Anti-CoV2 solutions were added to achieve varying concentrations in well between 0.89 and 18.4 nM.

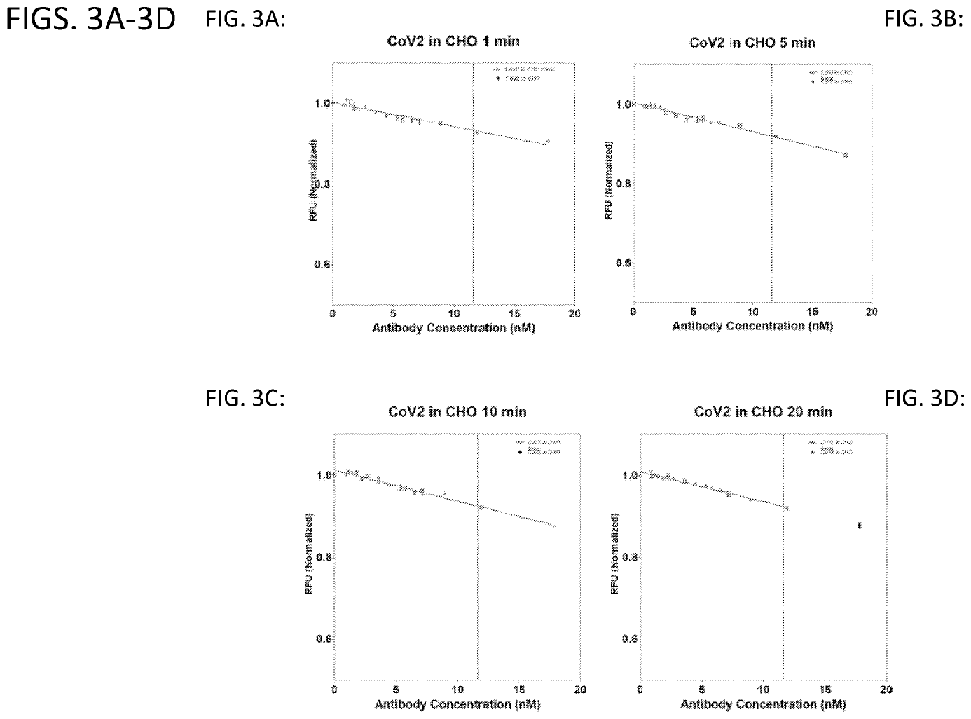

The results provided in FIGS. 3A-3D demonstrate SARS-CoV-2 nucleocapsid antibody detection in CHO cell culture fluid across the timepoints indicated using the DARQ assays of the present disclosure. Aliquots of 230 μL of ^{Rh}PrL/^{FL}CoV2 stock solution in CHO to achieve a final concentration in each well of 23.2 nM for both ^{Rh}PrL and ^{Fl}COV2. Aliquots of 20 μL Anti-CoV2 solutions were added to achieve varying concentrations in well between 0.89 and 18.4 nM.

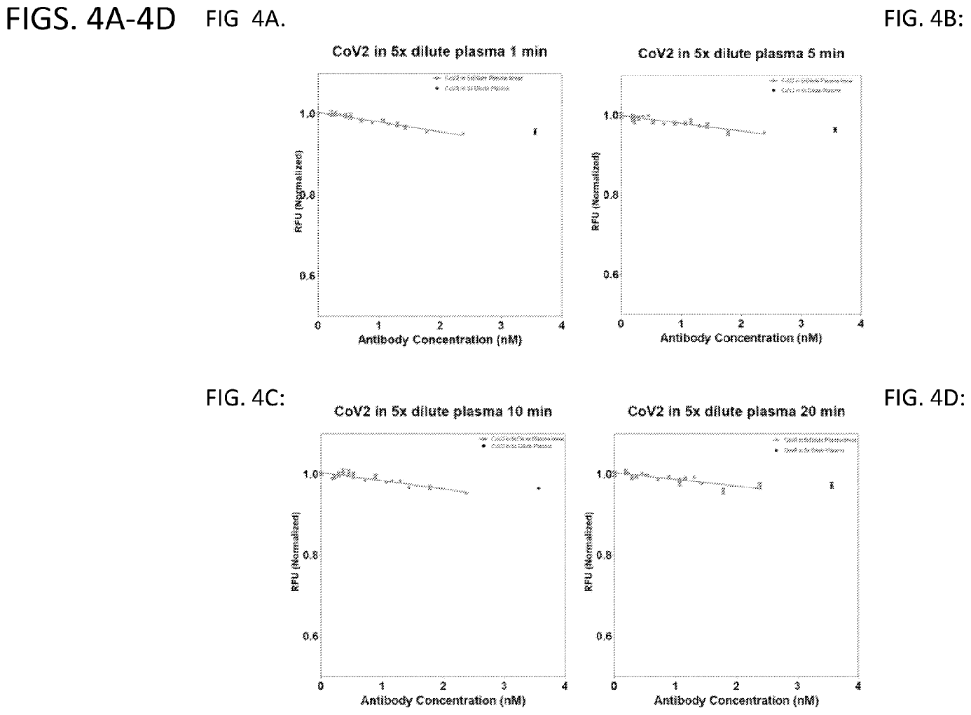

The results provided in FIGS. 4A-4D demonstrate SARS-CoV-2 nucleocapsid antibody detection in plasma samples across the timepoints indicated using the DARQ assays of the present disclosure. Aliquots of 230 μL of ^{Rh}PrL/^{FL}CoV2 stock solution in plasma to achieve a final concentration in each well of 23.2 nM for both ^{Rh}PrL and ^{Fl}COV2. Aliquots of 20 μL Anti-CoV2 solutions were added to achieve varying concentrations in well between 0.89 and 4.00 nM. In this experiment, the concentrations of antibody in the well were lowered by making a 5× dilution of the plasma to account for the binding of other IgG.

### 3. Compositions and Kits

Embodiments of the present disclosure also include a composition for performing a ratiometric bioassay to detect a target glycoprotein in a sample, as described above. In accordance with these embodiments, the composition includes a lectin coupled to a first fluorophore, a protein binding moiety coupled to a second fluorophore, and a sample comprising or suspected of comprising a target glycoprotein. The lectin coupled to the first fluorophore, the protein binding moiety coupled to the second fluorophore, and the sample can be combined in any suitable vessel, container, tube, and the like that allows for a binding complex to form. In some embodiments, emission from the first and the second fluorophore is altered upon exposure to light comprising an excitation wavelength of the first and/or the second fluorophore. In some embodiments, the altered fluorescence emission is proportional to the glycoprotein concentration in the sample. In some embodiments, the composition can be used to determine the concentration of the target glycoprotein in the sample based on the altered emission of the first and the second fluorophore. In some embodiments, the altered emission comprises decreased emission, increased emission, and/or a change in emission lifetime.

In some embodiments, the lectin is capable of binding a glycan epitope on the target glycoprotein in the sample. In some embodiments, the protein binding moiety is capable of binding a region of the target glycoprotein that does not comprise the glycan epitope. In some embodiments, the protein binding moiety comprises Protein L, Protein A, Protein G, or a protein-binding fragment thereof. In some embodiments, the first fluorophore is a high-energy fluorophore and the second fluorophore is a low-energy fluorophore. In some embodiments, the second fluorophore is a high-energy fluorophore and the first fluorophore is a low-energy fluorophore.

In some embodiments, the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532. In some embodiments, the low-energy fluorophore is selected from the group consisting of Rhodamine, Rhodamine B. Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

In some embodiments, the lectin is coupled to the first fluorophore at a lectin-to-fluorophore ratio of about 1:0.5 to about 1:20. In some embodiments, the protein binding moiety is coupled to the second fluorophore at an moiety-to-fluorophore ratio of about 1:0.2 to about 1:20. In some embodiments, the first fluorophore and the second fluorophore are present in the composition at a first-to-second fluorophore ratio of about 1:1 to about 1:20.

In some embodiments, the sample is undiluted. In some embodiments, the sample is a whole blood sample, a plasma sample, a serum sample, a urine sample, a saliva sample, a tissue sample, a plant sample, a soil sample, a water sample, a cell culture sample, a cell lysate sample, of a cell culture media. In some embodiments, the target glycoprotein is an antibody, a blood protein, a mammalian cell surface protein, a growth factor, or a protein from a pathogenic organism.

In some embodiments, a sample refers to any fluid sample containing or suspected of containing a target glycoprotein. The sample may be derived from any suitable source. In some cases, the sample may comprise a liquid, fluent particulate solid, or fluid suspension of solid particles. In some cases, the sample may be processed prior to the analysis described herein. For example, the sample may be separated or purified from its source prior to analysis; however, in certain embodiments, an unprocessed sample containing at least one target glycoprotein may be assayed directly. In a particular example, the source of a target glycoprotein is a mammalian (e.g., human) bodily substance (e.g., bodily fluid, blood such as whole blood (including, for example, capillary blood, venous blood, etc.), serum, plasma, urine, saliva, sweat, sputum, semen, mucus, lacrimal fluid, lymph fluid, amniotic fluid, interstitial fluid, lower respiratory specimens such as, but not limited to, sputum, endotracheal aspirate or bronchoalveolar lavage, cerebrospinal fluid, feces, tissue, organ, one or more dried blood spots, or the like). Tissues may include, but are not limited to oropharyngeal specimens, nasopharyngeal specimens, skeletal muscle tissue, liver tissue, lung tissue, kidney tissue, myocardial tissue, brain tissue, bone marrow, cervix tissue, skin, etc. The sample may be a liquid sample or a liquid extract of a solid sample. In certain cases, the source of the sample may be an organ or tissue, such as a biopsy sample, which may be solubilized by tissue disintegration/cell lysis. Additionally, the sample can be a nasopharyngeal or oropharyngeal sample obtained using one or more swabs that, once obtained, is placed in a sterile tube containing a virus transport media (VTM) or universal transport media (UTM), for testing.

A wide range of volumes of the fluid sample may be analyzed. In a few exemplary embodiments, the sample volume may be about 0.5 nL, about 1 nL, about 3 nL, about 0.01 μL, about 0.1 μL, about 1 μL, about 5 μL, about 10 μL, about 100 μL, about 1 mL, about 5 mL, about 10 mL, or the like. In some cases, the volume of the fluid sample is between about 0.01 μL and about 10 mL, between about 0.01 μL and about 1 mL, between about 0.01 μL and about 100 μL, or between about 0.1 μL and about 10 μL. In some cases, the fluid sample may be diluted prior to use in an assay. For example, in embodiments where the source containing a target glycoprotein is a human body fluid (e.g., blood, serum), the fluid may be diluted with an appropriate solvent (e.g., a buffer such as PBS buffer). A fluid sample may be diluted about 1-fold, about 2-fold, about 3-fold, about 4-fold, about 5-fold, about 6-fold, about 10-fold, about 100-fold, or greater, prior to use. In other cases, the fluid sample is not diluted prior to use in an assay.

Embodiments of the present disclosure also include a kit for performing a ratiometric bioassay to detect a target glycoprotein in a sample. In accordance with these embodiments, the kit includes a lectin coupled to a first fluorophore, a protein binding moiety coupled to a second fluorophore, and at least one container. In some embodiments, the kit further comprises a buffer and/or instructions for performing the bioassay. In some embodiments, the kit further comprises the target glycoprotein or a fragment or derivative thereof.

In some embodiments, the kit comprises at least one component for assaying the test sample for a target glycoprotein and corresponding instructions. Instructions included in kits can be affixed to packaging material or can be included as a package insert. While the instructions are typically written or printed materials they are not limited to such. Any medium capable of storing such instructions and communicating them to an end user is contemplated by this disclosure. Such media include, but are not limited to, electronic storage media (e.g., magnetic discs, tapes, cartridges, chips), optical media (e.g., CD ROM), and the like. As used herein, the term “instructions” can include the address of an internet site that provides the instructions.

The kit can also comprise a calibrator or control (e.g., purified, and optionally lyophilized, target glycoprotein or fragment thereof) and/or at least one container (e.g., tube, microtiter plates) for conducting the assay, and/or a buffer, such as an assay buffer or a wash buffer, either one of which can be provided as a concentrated solution, a substrate solution for the detectable label, or a stop solution. In some embodiments, the kit comprises all components that are necessary to perform the assay (e.g., reagents, standards, buffers, diluents, and the like). The instructions also can include instructions for generating a standard curve.

The kit may further comprise reference standards for quantifying a target glycoprotein. The reference standards may be employed to establish standard curves for interpolation and/or extrapolation of antibody concentrations. Standards cans include proteins or peptide fragments composed of amino acids residues or labeled proteins or peptide fragments for various analytes, as well as standards for sample processing, including standards involving spikes in proteins and quantitative peptides. Kits can also include quality control components (for example, sensitivity panels, calibrators, and positive controls). Preparation of quality control reagents is well-known in the art and is described on insert sheets for a variety of products. Sensitivity panel members can be used to establish assay performance characteristics, and can be useful indicators of the integrity of bioassay kit reagents, and the standardization of assays. The kit can also include other reagents required to conduct a bioassay or facilitate quality control evaluations, such as buffers, salts, enzymes, enzyme co-factors, substrates, detection reagents, and the like. Other components, such as buffers and solutions for the isolation and/or treatment of a test sample (e.g., pretreatment reagents), also can be included in the kit.

What is claimed is:

1. A ratiometric bioassay method for detecting a target antibody in a sample, the method comprising:
combining an antigen, or fragment thereof, coupled to a first fluorophore and an antibody binding moiety coupled to a second fluorophore with a sample comprising or suspected of comprising a target antibody;
exposing the antigen, or fragment thereof, coupled to the first fluorophore, the antibody binding moiety coupled to the second fluorophore, and the sample to light comprising an excitation wavelength of the first and/or the second fluorophore; and
detecting emission from the first and/or the second fluorophore;
wherein the emission from the first and the second fluorophore is altered, and wherein the altered emission is proportional to the antibody concentration in the sample.

2. The bioassay method according to claim 1, wherein the target antibody is capable of binding the antigen.

3. The bioassay method according to claim 1 or 2, wherein the antibody binding moiety is capable of binding a region of the target antibody that does not interfere with the antigen binding.

4. The bioassay method according to any of claims 1 to 3, wherein the antibody binding moiety comprises Protein L, Protein A, Protein G or an antibody-binding fragment thereof.

5. The bioassay method according to any of claims 1 to 4, wherein the first fluorophore is a high-energy fluorophore and the second fluorophore is a low-energy fluorophore.

6. The bioassay method according to any of claims 1 to 4, wherein the second fluorophore is a high-energy fluorophore and the first fluorophore is a low-energy fluorophore.

7. The bioassay method according to any of claims 1 to 6, wherein the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

8. The bioassay method according to any of claims 1 to 7, wherein the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

9. The bioassay method according to any of claims 1 to 8, wherein the antibody binding moiety is coupled to Rhodamine.

10. The bioassay method according to any of claims 1 to 9, wherein the antigen is coupled to fluorescein.

11. The bioassay method according to any of claims 1 to 10, wherein the method further comprises determining the concentration of the target antibody in the sample based on the altered emission of the first and the second fluorophore.

12. The bioassay method according to any of claims 1 to 11, wherein the altered emission comprises decreased emission, increased emission, and/or a change in emission lifetime.

13. The bioassay method according to any of claims 1 to 12, wherein the method further comprises incubating the sample comprising or suspected of comprising the target antibody, the antigen coupled to the first fluorophore, and the antibody binding moiety coupled to the second fluorophore for 30 minutes or less prior to measuring the emission of the first and/or the second fluorophore.

14. The bioassay method according to any of claims 1 to 13, wherein the antigen is coupled to the first fluorophore at an antigen-to-fluorophore ratio of about 1:0.2 to about 1:20.

15. The bioassay method according to any of claims 1 to 14, wherein the antibody binding moiety is coupled to the second fluorophore at a moiety-to-fluorophore ratio of about 1:0.2 to about 1:20.

16. The bioassay method according to any of claims 1 to 15, wherein the first fluorophore and the second fluorophore are present in the composition at a first-to-second fluorophore ratio of about 1:1 to about 1:20.

17. The bioassay method according to any of claims 1 to 16, wherein the sample is undiluted.

18. The bioassay method according to any of claims 1 to 17, wherein the sample is a whole blood sample, a plasma sample, a serum sample, a urine sample, a saliva sample, or a tissue sample.

19. The bioassay method according to any of claims 1 to 17, wherein the sample is a plant sample, a soil sample, a water sample, a cell culture sample, a cell lysate sample, or a cell culture media sample.

20. The bioassay method according to any of claims 1 to 19, wherein the antigen is from a pathogenic organism.

21. The bioassay method according to claim 20, wherein the pathogenic organism is a bacteria, a virus, or a fungus.

22. The bioassay method according to any of claims 1 to 21, wherein the antigen is from SARS-CoV-2.

23. A composition for performing a ratiometric bioassay to detect a target glycoprotein in a sample, the composition comprising:
an antigen coupled to a first fluorophore;
an antibody binding moiety coupled to a second fluorophore; and
a sample comprising or suspected of comprising a target antibody;
wherein emission from the first and the second fluorophore is altered upon exposure to light comprising an excitation wavelength of the first and/or the second fluorophore, and wherein the altered emission is proportional to the target glycoprotein concentration in the sample.

24. The composition according to claim 23, wherein the target antibody is capable of binding the antigen.

25. The composition according to claim 23 or claim 24, wherein the antibody binding moiety is capable of binding a region of the target antibody that does not interfere with the antigen binding.

26. The composition according to any of claims 23 to 25, wherein the antibody binding moiety comprises Protein L, Protein A, Protein G or an antibody-binding fragment thereof.

27. The composition according to any of claims 23 to 26, wherein the first fluorophore is a high-energy fluorophore and the second fluorophore is a low-energy fluorophore.

28. The composition according to any of claims 23 to 27, wherein the second fluorophore is a high-energy fluorophore and the first fluorophore is a low-energy fluorophore.

29. The bioassay method according to any of claims 23 to 28, wherein the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

30. The composition according to any of claims 23 to 29, wherein the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

31. The composition according to any of claims 23 to 30, wherein the altered emission comprises decreased emission, increased emission, and/or a change in emission lifetime.

32. The composition according to any of claims 23 to 31, wherein the antigen is coupled to the first fluorophore at an antigen-to-fluorophore ratio of about 1:0.5 to about 1:20.

33. The composition according to any of claims 23 to 32, wherein the antibody binding moiety is coupled to the second fluorophore at a moiety-to-fluorophore ratio of about 1:0.2 to about 1:20.

34. The composition according to any of claims 23 to 33, wherein the first fluorophore and the second fluorophore are present in the composition at a first-to-second fluorophore ratio of about 1:1 to about 1:20.

35. The composition according to any of claims 23 to 34, wherein the sample is undiluted.

36. The composition according to any of claims 23 to 35, wherein the sample is a whole blood sample, a plasma sample, a serum sample, a urine sample, a saliva sample, a tissue sample, a plant sample, a soil sample, a water sample, a cell culture sample, a cell lysate sample, of a cell culture media.

37. The composition according to any of claims 23 to 36, wherein the antigen is from a pathogenic organism.

38. The composition according to any of claims 23 to 36, wherein the antigen is from SARS-CoV-2.

39. A kit for performing a ratiometric bioassay to detect a target glycoprotein in a sample, the kit comprising:
an antigen coupled to a first fluorophore;
an antibody binding moiety coupled to a second fluorophore; and
at least one container.

40. The kit according to claim 39, wherein the kit further comprises a buffer and/or instructions for performing the bioassay.

41. The kit according to claim 39 or claim 40, wherein the kit further comprises the target antibody, or a fragment or derivative thereof.

42. A ratiometric bioassay method for detecting a target glycoprotein in a sample, the method comprising:
combining a lectin or fragment thereof coupled to a first fluorophore, a protein binding moiety coupled to a second fluorophore, and a sample comprising or suspected of comprising a target glycoprotein;
exposing the lectin coupled to the first fluorophore, the protein binding moiety coupled to the second fluorophore, and the sample to light comprising an excitation wavelength of the first and/or the second fluorophore; and
detecting emission from the first and/or the second fluorophore;
wherein the emission from the first and the second fluorophore is altered, and wherein the altered emission is proportional to the protein concentration in the sample.

43. The bioassay method according to claim 42, wherein the lectin is capable of binding a glycan epitope on the target glycoprotein in the sample.

44. The bioassay method according to claim 42 or 43, wherein the protein binding moiety is capable of binding a region of the target glycoprotein that does not comprise the glycan epitope.

45. The bioassay method according to any of claims 42 to 44, wherein the protein binding moiety comprises Protein L, Protein A, Protein G or a protein-binding fragment thereof.

46. The bioassay method according to any of claims 42 to 45, wherein the lectin is selected from the group consisting of LTL (Lotus tetragonolobus), PSA (Pisum sativum), LCA (Lens culinaris), UEA-I (Ulex europaeus), AOL (Aspergillus oryzae), AAL (Aleuria aurantia), MAL_I (Maackia amurensis), SNA (Sambucus nigra), SSA (Sambucus sieboldiana), TJA-I (Trichosanthes japonica), PHAL (Phaseolus vulgaris), ECA (Erythrina cristagalli), RCA120 (Ricinus communis), PHAE (Phaseolus vulgaris), DSA (Datura stramonium), GSL-II (Griffonia simplicifolia), NPA (Narcissus pseudonarcissus), ConA (Canavalia ensiformis), GNA (Galanthus nivalis), HHL (Hippeastrum hybrid), ACG (mushroom, Agrocybe cylindracea), TxLCI (Tulipa gesneriana), BPL (Bauhinia purpurea), TJA-II (Tanthes japonica), EEL (Euonymus europaeus), ABA (fungus, Agaricus bisporus), LEL (tomato, Lycopersicon esculentum), STL (potato, Solanum tuberosum), UDA (Urtica dioica), PWM (pokeweed, Phytolacca Americana), Jacalin (Artocarpus integrifolia), PNA (peanut, Arachis hypogaea), WFA (Wisteria floribunda), ACA (Amaranthus caudatus), MPA (Maclura pomifera), HPA (snail, Helix pomatia), VVA (Vicia villosa), DBA (Dolichos biflorus), SBA (soybean, Dolichos biflorus), Calsepa (Calystegia sepium), PTL-I (Psophocarpus tetragonolobus), MAH (Maackia amurensis), WGA (wheat germ, Triticum aestivum), GSL-I A4 (Griffonia simplicifolia), and GSL-I B4 (Griffonia simplicifolia), or a glycan epitope binding fragment thereof.

47. The bioassay method according to any of claims 42 to 46, wherein the first fluorophore is a high-energy fluorophore and the second fluorophore is a low-energy fluorophore.

48. The bioassay method according to any of claims 42 to 47, wherein the second fluorophore is a high-energy fluorophore and the first fluorophore is a low-energy fluorophore.

49. The bioassay method according to any of claims 42 to 48, wherein the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

50. The bioassay method according to any of claims 42 to 49, wherein the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

51. The bioassay method according to any of claims 42 to 50, wherein the lectin is coupled to Rhodamine.

52. The bioassay method according to any of claims 42 to 51, wherein the protein binding moiety is coupled to fluorescein.

53. The bioassay method according to any of claims 42 to 52, wherein the method further comprises determining the concentration of the target glycoprotein in the sample based on the altered emission of the first and the second fluorophore.

54. The bioassay method according to any of claims 42 to 53, wherein the altered emission comprises decreased emission, increased emission, and/or a change in emission lifetime.

55. The bioassay method according to any of claims 42 to 54, wherein the method further comprises incubating the sample comprising or suspected of comprising the target glycoprotein, the lectin coupled to the first fluorophore, and the protein binding moiety coupled to the second fluorophore for 30 minutes or less prior to measuring the emission of the first and/or the second fluorophore.

56. The bioassay method according to any of claims 42 to 55, wherein the lectin is coupled to the first fluorophore at a lectin-to-fluorophore ratio of about 1:0.2 to about 1:20.

57. The bioassay method according to any of claims 42 to 56, wherein the protein binding moiety is coupled to the second fluorophore at a moiety-to-fluorophore ratio of about 1:0.2 to about 1:20.

58. The bioassay method according to any of claims 42 to 57, wherein the first fluorophore and the second fluorophore are present in the composition at a first-to-second fluorophore ratio of about 1:1 to about 1:20.

59. The bioassay method according to any of claims 42 to 58, wherein the sample is undiluted.

60. The bioassay method according to any of claims 42 to 59, wherein the sample is a whole blood sample, a plasma sample, a serum sample, a urine sample, a saliva sample, or a tissue sample.

61. The bioassay method according to any of claims 42 to 60, wherein the sample is a plant sample, a soil sample, a water sample, a cell culture sample, a cell lysate sample, or a cell culture media sample.

62. The bioassay method according to any of claims 42 to 61, wherein the target glycoprotein is an antibody, a blood protein, a mammalian cell surface protein, or a growth factor.

63. The bioassay method according to any of claims 42 to 62, wherein the target glycoprotein is from a bacteria, virus, or fungus.

64. A composition for performing a ratiometric bioassay to detect a target glycoprotein in a sample, the composition comprising:
a lectin coupled to a first fluorophore;
a protein binding moiety coupled to a second fluorophore; and
a sample comprising or suspected of comprising a target glycoprotein;
wherein emission from the first and the second fluorophore is altered upon exposure to light comprising an excitation wavelength of the first and/or the second fluorophore, and wherein the altered emission is proportional to the target glycoprotein concentration in the sample.

65. The composition according to claim 64, wherein the lectin is capable of binding a glycan epitope on the target glycoprotein in the sample.

66. The composition according to claim 64 or claim 65, wherein the protein binding moiety is capable of binding a region of the target glycoprotein that does not comprise the glycan epitope.

67. The composition according to any of claims 64 to 66, wherein the protein binding moiety comprises Protein L, Protein A, Protein G, or a protein-binding fragment thereof.

68. The composition according to any of claims 64 to 67, wherein the first fluorophore is a high-energy fluorophore and the second fluorophore is a low-energy fluorophore.

69. The composition according to any of claims 64 to 68, wherein the second fluorophore is a high-energy fluorophore and the first fluorophore is a low-energy fluorophore.

70. The bioassay method according to any of claims 64 to 69, wherein the high-energy fluorophore is selected from the group consisting of Fluorescein, Oregon Green 488, Oregon Green 514, Rhodamine Green, Rhodamine Green-X, Eosin, 4′, 6-diamidino-2-phenylindole, Alexafluor 405, Alexafluor 350, Alexafluor 500, Alexafluor 488, Alexafluor 430, Alexafluor 514, and Alexafluor 532.

71. The composition according to any of claims 64 to 70, wherein the low-energy fluorophore is selected from the group consisting of Rhodamine, Rhodamine B, Rhodamine Red-X, Tetramethylrhodamine, Lissamine, Texas Red and Texas Red-X, Naphthofluorescein, Carboxyrhodamine 6G, Alexafluor 555, Alexafluor 546, Alexafluor 568, Alexafluor 594, Alexafluor 610, Alexafluor 633, Alexafluor 635, Alexafluor 647, Alexafluor 660, Alexafluor 680, Alexafluor 700, and Alexafluor 750.

72. The composition according to any of claims 64 to 71, wherein the altered emission comprises decreased emission, increased emission, and/or a change in emission lifetime.

73. The composition according to any of claims 64 to 72, wherein the lectin is coupled to the first fluorophore at a lectin-to-fluorophore ratio of about 1:0.5 to about 1:20.

74. The composition according to any of claims 64 to 73, wherein the protein binding moiety is coupled to the second fluorophore at an moiety-to-fluorophore ratio of about 1:0.2 to about 1:20.

75. The composition according to any of claims 64 to 74, wherein the first fluorophore and the second fluorophore are present in the composition at a first-to-second fluorophore ratio of about 1:1 to about 1:20.

76. The composition according to any of claims 64 to 75, wherein the sample is undiluted.

77. The composition according to any of claims 64 to 76, wherein the sample is a whole blood sample, a plasma sample, a serum sample, a urine sample, a saliva sample, a tissue sample, a plant sample, a soil sample, a water sample, a cell culture sample, a cell lysate sample, of a cell culture media.

78. The composition according to any of claims 64 to 77, wherein the target glycoprotein is an antibody, a blood protein, a mammalian cell surface protein, a growth factor, or a protein from a pathogenic organism.

79. A kit for performing a ratiometric bioassay to detect a target glycoprotein in a sample, the kit comprising:
a lectin coupled to a first fluorophore;
a protein binding moiety coupled to a second fluorophore; and
at least one container.

80. The kit according to claim 79, wherein the kit further comprises a buffer and/or instructions for performing the bioassay.

81. The kit according to claim 79 or claim 80, wherein the kit further comprises the target glycoprotein or a fragment or derivative thereof.
